(19) United States
(12) Patent Application Publication
(10) Pub. No.: US 2023/0293350 A1
(43) Pub. Date: Sep. 21, 2023

(54) APPLANATION DEVICE INTENDED TO BE COUPLED TO AN OPTHALMOLOGICAL LASER SURGERY SYSTEM

(71) Applicants: UNIVERSITE DE BORDEAUX, BORDEAUX (FR); CENTRE NATIONAL DE LA RECHERCHE SCIENTIFIQUE - CNRS, PARIS (FR); CENTRE HOSPITALIER UNIVERSITAIRE DE BORDEAUX, TALENCE (FR)

(72) Inventors: John LOPEZ, GRADIGNAN (FR); Donetsi IRIBARREN, FLOIRAC (FR); David TOUBOUL, BORDEAUX (FR)

(21) Appl. No.: 18/006,198

(22) PCT Filed: Jul. 12, 2021
(86) PCT No.: PCT/FR2021/051288

§ 371 Date: Jan. 20, 2023

(30) Foreign Application Priority Data
Jul. 22, 2020 (FR) FR2007710

(51) Int. Cl. A61F 9/009

(52) U.S. Cl. CPC A61F 9/009; A61B 2218/007; A61F 2009/0087

(57) ABSTRACT

An applanation device designed to be positioned between the eye of a patient and a laser system, to hold the eye in position and create a reference plane for an ophthalmological laser surgery operation, the device comprising: an applanation cone defining an inner space, an upper opening and a lower opening; the applanation cone comprising an upper portion configured to be secured to a projecting portion of a focusing optical unit of the laser system and a lower portion comprising suction means; the lower opening being defined by a lower rim; an applanation glass that is transparent to the laser beam generated by the laser system and is positioned and held by the suction means against the lower rim so as to close the lower opening.

### CROSS REFERENCE TO RELATED APPLICATONS

This application is a National Stage of International Application No. PCT/FR2021/051288, having an International Filing Date of 12 Jul. 2021, which designated the United States of America, and which International Application was published under PCT Article 21 (2) as WO 2022/018349A1, which claims priority from and the benefit of French Pat. Application No. 2007710 filed on 22 Jul. 2020, the disclosures of which are incorporated herein by reference in their entireties.

### BACKGROUND

### Field

The disclosure relates to the field of ocular surgery appliances. More specifically, the disclosure relates to an applanation device intended to flatten the eye of the patient and to hold it in position during a laser ophthalmological surgery operation.

The disclosure relates also to an ophthalmological surgery appliance intended for example for performing corneal grafts and ocular refractive surgery equipped with an associated applanation device.

### Brief Description of Developments

The cornea forms part of the peripheral tunic of the eye. It is a transparent dielectric medium like glass cut in dome form with faces almost parallel forming a biconvex aspherical diopter. Its average diameter is approximately 12 mm and its central average thickness is approximately 540 microns. It has an average radius of curvature of the order of 7.8 mm. It makes it possible to make the incident light rays converge toward the retina to form an image thereon.

Under the effect of an accident in life or a specific pathology, the cornea can become partially or totally opaque, thus degrading the vision. A corneal graft then constitutes the only effective treatment to restore the function of the sick cornea. It also makes it possible to reestablish good visual acuity and to eliminate the pains provoked by corneal injuries. Keratoplasty, or corneal grafting, generally consists in extracting a part or all of a pathological cornea, and in replacing it with a healthy cornea, coming from a donor.

It is also possible to use refractive surgery to compensate for the vision defects, called ametropias, reflecting a failure of focusing of the light rays on the plane of the retina through the retina. The operable ametropias are myopia, hypermetropy, astigmatism and presbyopia. Lasers are currently used to assist the surgeon in treating the healthy cornea to correct vision defects.

An example that can be cited here is the LASIK technique which consists in superficially cutting a flap of cornea by a thickness of 90 to 120 microns using a mechanical microkeratome (blade) or a femtosecond laser to then raise this flap for the time it takes to remodel the underlying cornea. The Excimer type ArF laser with a 193 nm ultraviolet radiation and a pulse duration of 10 to 25 ns allows a very accurate remodeling (approximately 0.25 micron of photoablation per pulse). This procedure is the most widely practiced currently for treating myopia.

Through the significant developments in laser technology, laser ocular surgery has become a technique of choice in ophthalmological surgical applications.

During the operation, in order to ensure an accurate coupling between the laser beam and a region of the eye to be treated by the laser beam, the eye of the patient must be held in a fixed position with respect to the laser beam so as to avoid any movement of the eye with respect to the focal point of the laser beam which could induce non-optimal results or irreversible damage to the eye.

It is known practice to use an applanation device which is designed to come into contact with the cornea and make it possible to flatten the surface thereof during the operation to allow the laser beam to be focused at a chosen depth. It generally comprises an applanation cone and a suction ring. The applanation cone comprises one end coupled to a terminal end of the laser system and another end provided with an applanation glass. The applanation glass is held in position, for example by gluing on the perimeter of the end of the applanation cone. During the operation, the suction ring is engaged and centered by the surgeon at the corneal limbus. A suction system linked to the suction ring makes it possible to ensure immobilization of the eye. The applanation cone is displaced via the terminal of the laser system to be engaged in the suction ring such that the applanation glass is in contact with the eye and subjects the eye to the curvature of the applanation lens during the treatment. The applanation glass thus forms a reference plane for the focusing of the laser beam.

### Technical Problem

According to the known applanation devices, the applanation glass is generally fixed to the applanation cone by a layer of glue whose thickness is not uniform over the entire surface of the fixing. Furthermore, from one applanation device to another, the thickness is not reproducible. This thickness variation can induce errors in the positioning of the reference plane formed by the applanation glass with respect to the focusing system. Now, it is essential for the applanation glass to be positioned at a predetermined and precise distance from the focusing system of the laser system to be able to focus the spot of the beam at a chosen depth. Consequently, in the proposed solutions, it is necessary to perform a check on the distance between the optical output of the focusing optical system and the reference plane formed by the applanation glass for each applanation cone before the start of the operation. This check results either in the applanation cone being changed or this distance being corrected, in order to compensate any inaccuracies of depth of focusing, rendering the operation longer and more complex. The deposition of the glue can also induce a defect of uniformity or of thickness on the glue joint which would lead to a defect of horizontality of the applanation glass. Furthermore, the glue can be subject to shrinkage linked to the evaporation of the solvent, potentially embrittling the fixing of the applanation glass. Finally, the glue deposits can also be found on the functional part of the applanation glass, in the laser passage, thus rendering the part formed by the applanation cone and the applanation glass non-conformal for use.

According to another drawback, the applanation cone is generally of one-time use, because of the permanent fixing of the applanation glass on the cone. It is therefore necessary to replace the cone and glass assembly on each operation.

According to yet another drawback, the known applanation devices do not generally offer the possibility of readjusting the distance between the applanation glass and the focusing system.

One objective of the present disclosure is therefore to propose an applanation device which makes it possible to resolve the technical problems posed by the devices of the prior art, by developing a structure that is particularly designed to be coupled to a laser ophthalmological surgery system, that is easy and practical to use, that makes it possible to position the applanation glass with great accuracy on the eye, and also that is mobile in the direction of the laser beam to be able to easily adjust the distance between the applanation glass and the focusing system.

### SUMMARY

In order to remedy the abovementioned drawbacks of the state of the art, the present disclosure proposes an applanation device intended to be positioned between the eye of a patient and a laser system, to hold the eye in position and create a reference plane for a laser ophthalmological surgery operation, said device comprising:
an applanation cone defining an internal space, a top aperture and a bottom aperture;
said applanation cone comprising an upper part configured to be fixed onto a protruding part of a focusing optical block of the laser system and a lower part comprising suction means;
the bottom aperture being delimited by a bottom rim;
an applanation glass transparent to the laser beam generated by the laser system being positioned and held by said suction means against the bottom rim so as to close the bottom aperture.

According to one embodiment of the disclosure, the suction means comprise at least one suction channel extending in the wall of said applanation cone between a suction input and a plurality of suction orifices formed in the bottom rim.

According to one embodiment of the disclosure, the applanation device further comprises a fixing ring configured to be securely mounted on the protruding lower part of the focusing optical block, the upper part of said applanation cone being configured to be mounted with translational mobility in a direction parallel to the optical axis of the focusing optical block on said fixing ring.

According to another embodiment of the disclosure, the applanation device further comprises an angular orientation ring configured to be mounted with rotational mobility in a plane at right angles to the optical axis of the focusing optical block on said protruding part, the upper part of said applanation cone being configured to be mounted with translational mobility in a direction parallel to the optical axis of the focusing optical block on said angular orientation ring.

According to one embodiment of the disclosure, the applanation cone comprises at least one camera holding element fixed onto the outer wall of the applanation cone and at least one side aperture arranged with respect to said holding element so as to allow passage of an imaging beam generated by said at least one camera to view a cutting zone of the eye situated under the applanation glass.

Preferably, said holding element is configured such that the imaging beam generated by said camera is inclined by an angle of between 30° and 50°, preferably between 45° and 47°, with respect to an optical axis of symmetry of the focusing block.

The features set out in the following paragraphs can, optionally, be implemented. They can be implemented independently of one another or in combination with one another:
the applanation glass comprises at least one pattern or a graduated rule produced on the surface or in the volume;
the applanation glass has a planar-planar or planar-concave geometry;
the applanation cone is produced in a metal material by additive manufacturing.

According to another embodiment of the disclosure, the applanation cone comprises an axial displacement ring provided with an internal threading, designed to be engaged by screwing with an external threading produced on the outer wall of the fixing ring or of the orientation ring, said axial displacement ring ensuring, in a screwing step, an axial displacement of said applanation cone.

According to another embodiment of the disclosure, the applanation device further comprises a locking ring configured to lock the applanation cone in position.

According to an exemplary embodiment, the applanation cone comprises a lower part fixed onto the upper part, said lower part having an upside-down truncated cone form.

The disclosure also proposes an applanation assembly for holding the eye of a patient in position and creating a reference plane for a laser system for a laser ophthalmological surgery operation, said assembly comprising:
an applanation device as defined above;
a suction ring designed to come into contact with a surface of the eye of the patient;
a portion of said lower part of the applanation cone being configured to be inserted into the ring to position the applanation glass inside the suction ring so as to come into contact with the eye of the patient.

A subject of the disclosure is also a laser ophthalmological surgery appliance for producing a cut in an ocular biological tissue, such as a cornea or a lens, comprising:
a laser unit designed to deliver a laser beam;
a focusing optical block for focusing the laser beam at a focal point in the ocular biological tissue;
said focusing optical block comprising a protruding lower part extending from a bottom surface of the laser unit;
an applanation device as defined above configured to be fixed onto the protruding lower part of the focusing optical block so as to position the applanation glass at a distance substantially close to the focusing distance with respect to the optical output of the focusing optical block.

According to one embodiment of the disclosure, the appliance further comprises at least one camera configured to view the cutting zone, said at least one camera being held in position by one of the camera holding elements fixed onto the outer wall of the applanation cone.

According to another embodiment of the disclosure, the appliance further comprises a centering camera configured for the centering of the optical axis of symmetry of the focusing system with respect to the biological tissue, one end of the centering camera being inserted into the applanation cone via one of the two apertures provided in the applanation cone.

### BRIEF DESCRIPTION OF THE FIGURES

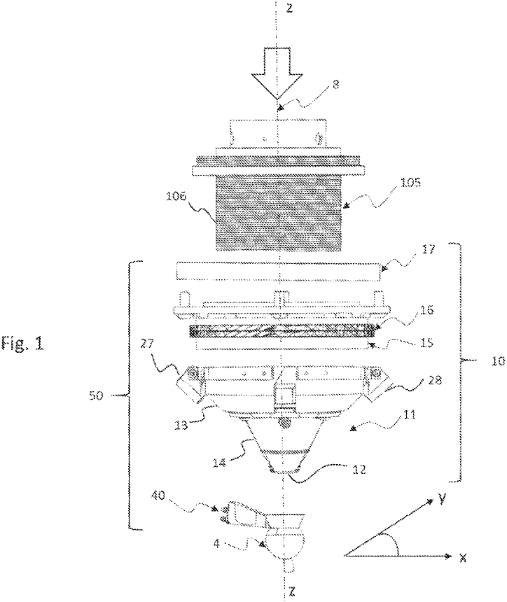

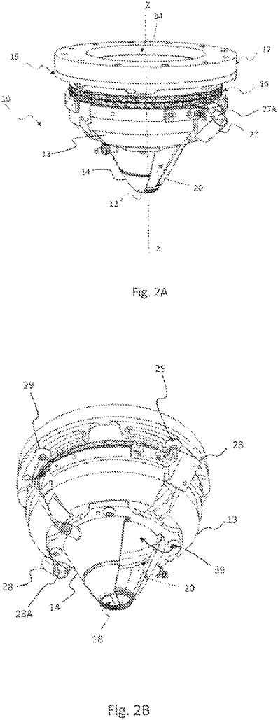

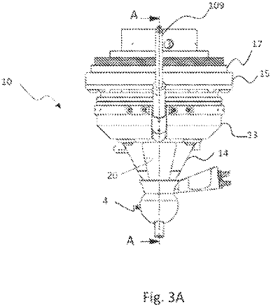

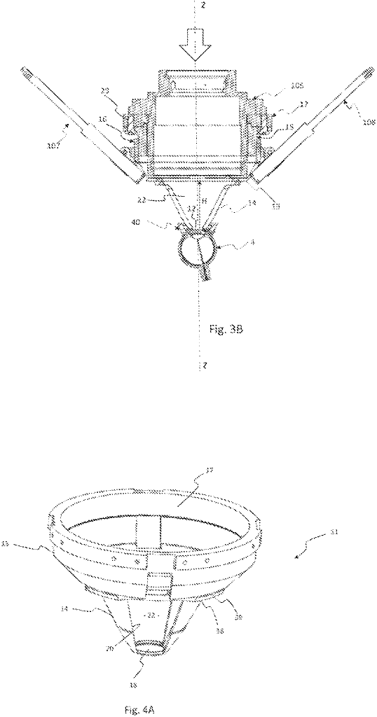

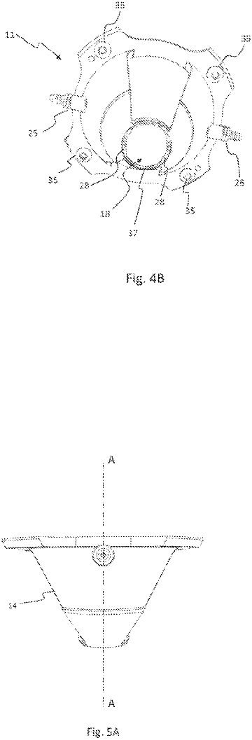

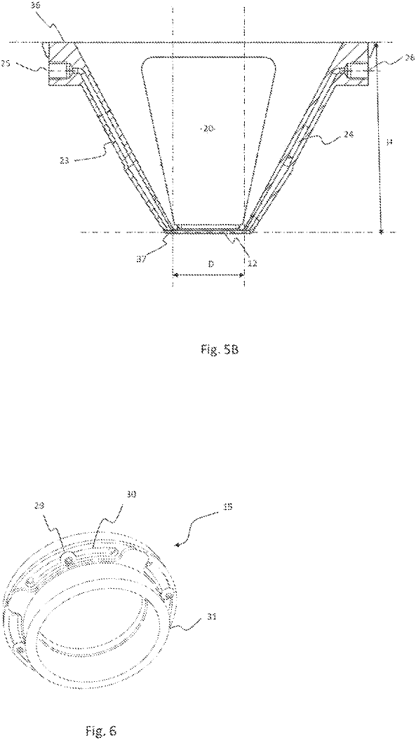

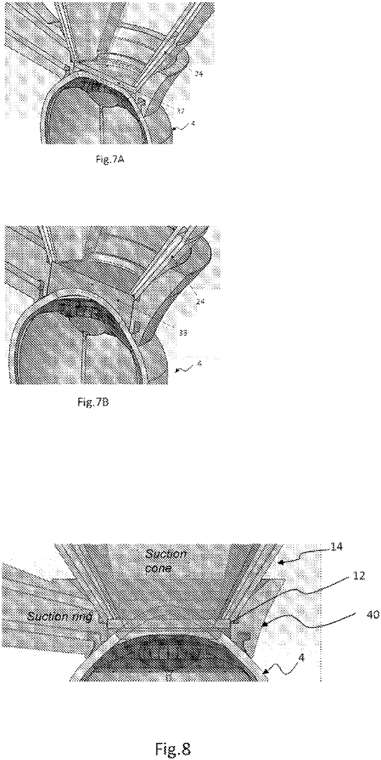

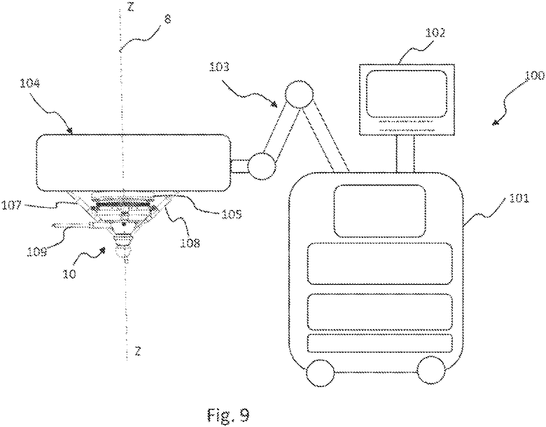

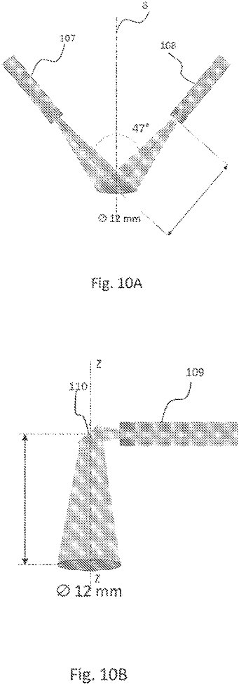

Other features, details and advantages of the disclosure will become apparent on reading the following detailed description, and on analyzing the attached drawings, in which:

[FIG. 1] represents an exploded view of the applanation device according to an embodiment of the disclosure, positioned between a focusing optical block and a suction ring placed above an eye;

[FIG. 2A] represents a perspective view of the applanation device in an assembled configuration;

[FIG. 2B] represents a perspective view from a bottom view of the device of FIG. 2A;

[FIG. 3A] represents a profile view according to the view from one of the two side vision cameras of the applanation device;

[FIG. 3B] represents a cross-sectional view of the applanation device on the line A-A in FIG. 3A;

[FIG. 4A] represents a perspective profile view of the applanation cone;

[FIG. 4B] represents a bottom perspective view of the applanation cone of FIG. 4A without the applanation glass;

[FIG. 5A] represents a profile view of the applanation cone;

[FIG. 5B] represents a cross-sectional view of the applanation cone on the line A-A in FIG. 5A showing the suction channels;

[FIG. 6] represents a perspective view of the angular orientation ring;

[FIG. 7A] represents an example of applanation by a planar-planar glass in position bearing against the surface of the cornea of the eye;

[FIG. 7B] represents an example of applanation by a planar-concave glass in position bearing against the surface of the cornea of the eye;

[FIG. 8] schematically represents a partial cross-sectional view of the assembly formed by a suction ring and a lower part of the applanation cone and the applanation glass applied to the cornea so as to shape the surface of the cornea to the geometry of the bottom surface of the applanation glass, whether it be planar or curved;

[FIG. 9] schematically represents an ophthalmological surgery appliance equipped with an applanation device according to an embodiment of the disclosure;

[FIG. 10A] schematically represents a front view of the arrangement of two side vision cameras;

[FIG. 10B] schematically represents a front view of the arrangement of the centering camera.

For greater clarity, similar elements have been identified by identical reference symbols throughout the figures.

### DETAILED DESCRIPTION

The drawings and the description hereinbelow contain, for the most part, elements of definite character. They will therefore be able not only to serve to give a better understanding of the present disclosure, but will also contribute to the definition thereof, as appropriate.

The present disclosure proposes an applanation device which makes it possible, in a laser ophthalmological surgery operation, by applying an applanation glass into contact with the eye to be treated, to stabilize the eye with respect to the focal point of the laser beam. This device also makes it possible to flatten the eye of the patient by means of the applanation glass so as to create a reference plane to position the focal point of the laser on the zone of the eye to be treated. The applanation device is designed to be coupled on the one hand to a suction ring positioned on the eye of a patient and, on the other hand, to a laser system dedicated to laser ophthalmological surgery. The device of the present disclosure, through a suction mechanism, allows the applanation glass to be positioned and held accurately, simply and reproducibly.

FIG. 1 schematically represents an applanation device 10 according to an embodiment. Here the applanation device is schematically represented in a context of use for a surgical intervention involving cutting the cornea. The device is represented here between a focusing optical block 105 and a suction ring 40 positioned on the eye 4. The focusing optical block 105 is disposed on the optical path of a laser beam delivered by a laser system not illustrated in the figure and designed to focus the laser beam at a focal point in the thickness of the cornea. The laser beam delivered by the laser system is represented here by an arrow toward the focusing optical block 105. An optical axis of symmetry 8 passing through the center of the cornea and perpendicular to the surface of the cornea extends in a direction Z-Z orthogonal to a plane (XY). The cornea, when it is flattened by the applanation glass, extends substantially in the plane (XY) . During the ophthalmological surgery operation, the incident laser beam is focused at different depths in volume in the cornea in a direction parallel to the axis Z with a normal angle of incidence.

According to an embodiment of the disclosure, the applanation device 10 comprises an applanation cone 11 and an applanation glass 12 held by suction on a lower part of the applanation cone 11, an angular orientation ring 15 and a fixing ring 17 which is securely mounted by screwing on a protruding part 106 of the focusing optical block 105 when the applanation device is coupled to the laser system.

Preferably, the applanation glass is made of a material that is transparent in the visible, the surface of which is of optical quality, chemically inert, and of great dimensional stability.

FIGS. 2A and 2B illustrate the device in an assembled configuration. The hollow suction cone 11 receives a bottom part of the angular orientation ring 15 which is itself fixed via fixing screws onto the fixing ring 17. In the configuration in which the applanation device is coupled to the laser system for an ophthalmological surgery operation, the fixing ring 17 is mounted securely on the protruding part 106 of the focusing optical block 105. When all the parts of the device are assembled, the device is defined by an internal space 39, a top aperture 34 and a bottom aperture 18 that is closed by the applanation glass 12 which is transparent to the wavelength of the laser beam. The axis of symmetry of the applanation device 10 substantially coincides with the optical axis of symmetry 8 of the cornea and of the focusing optical block 105.

According to a particularly advantageous embodiment, the applanation device further comprises two camera-holding elements 27, 28 fixed onto the outer wall of the applanation cone and two side apertures 20 produced in the wall of a lower part of the applanation cone 13.

The holding elements 27, 28 each comprise a tubular housing 27A, 28A configured to receive a vision camera 107, 108, called side vision cameras. As illustrated in FIGS. 10A and 10B, the cameras are oriented so as to generate an imaging beam inclined by an angle of between 30° and 50°, preferably between 45° and 47°, with respect to the optical axis of symmetry of the focusing block. The apertures 20 are thereby arranged with respect to the holding elements so as to allow passage of the imaging beam generated by the camera to view a cutting zone of the eye situated under the applanation glass. The apertures 20 are situated plumb with the holding elements 27, 28.

The cameras 107, 108 allow the surgeon to view the cornea on a screen during the operation. By virtue of the presence of the side apertures 20, it is possible to introduce a centering camera 109 (FIG. 9) into the applanation cone, the field of view of which is centered with respect to the optical axis of the cornea so as to correctly position the surgical appliance above the eye of the patient.

The centering camera 109 is movable in a transverse direction between a first position in which it is in the internal space of the applanation cone 11, arranged between the focusing system and the tissue to perform the centering, and a second position in which it is removed before the start of the operation.

When the applanation cone 11 is mounted and positioned on the angular orientation ring 15, it is necessary to ensure that one of the apertures 20 is opposite the centering camera 109. For that, and referring to FIG. 6, the orientation ring 15 is provided for example with four oblong holes 30 in which fixing screws 29 are arranged. After having adjusted the distance H between the optical output of the focusing block and the applanation glass by rotating the applanation cone 11 on the orientation ring 15 to generate an axial displacement on the axis Z-Z, it is possible to rotate the angular orientation ring 15 in the plane (XY) by displacing the fixing screws 29 in the oblong holes 30 so as to place one of the apertures 20 opposite the centering camera. The rotation in the plane (XY) does not induce any axial displacement on the axis Z-Z. At the end of this rotation, the fixing screws 29 are fixed onto the fixing ring 17.

Referring to FIGS. 3 and 4, the applanation cone 11 is a hollow body defined by an internal space 22, a top aperture 17 and a bottom aperture 18. The bottom aperture 18 is centered with respect to the top aperture 17. The applanation cone 11 comprises an upper part 13 and a lower part 14. The upper part 13 takes the form of a ring that has the form of an upside-down truncated cone defined by a substantially cylindrical internal space. It is provided on its inner wall with an internal threading. The dimension of the internal space is designed to receive a bottom part of the orientation ring 15 which has a cylindrical form. The internal threading and the external threading produced on the outer wall of the orientation ring cooperate together to displace the applanation body 11 on the axis Z-Z on the orientation ring 15 in order to adjust the distance H between the optical output of the focusing optical block 105 and the applanation glass 12. The distance H corresponds substantially to the working distance of the focusing optical block. It is generally between 20 mm and 70 mm, preferably between 30 and 50 mm. For the remainder of the description, the upper part 13 of the applanation cone is called axial displacement ring.

Referring to FIGS. 5A and 5B, the lower part 14 also has the form of an upside-down truncated cone. It comprises a top rim 36 and a bottom rim 37 delimiting the bottom aperture 18. The bottom rim 37 forms a suction surface to hold the applanation glass 12 which closes the bottom aperture 18. The applanation glass 12 has a substantially circular form and is produced in a material transparent to the wavelength of the laser beam delivered by the laser system. The diameter of the applanation glass is substantially equal to the diameter of the base of the truncated cone 14. The diameter of the bottom aperture D forms the diameter of the working field of the laser beam which is approximately 10 mm at the level of the eye of the patient.

The lower part 14 comprises suction means for holding the applanation glass against the bottom rim. According to an exemplary embodiment illustrated in FIG. 5B, they comprise two suction channels 23, 24 formed in the wall of the lower part of the suction body. Hereinafter in the description, the lower part is designated by the term “suction cone”. Each channel 23, 24 extends between a suction input 25, 26 and a suction output situated in the bottom rim 37. According to an exemplary embodiment and referring to FIG. 4B, the suction output is formed by a plurality of suction orifices 28 situated in the bottom rim 37. The two main channels 23, 24 are therefore divided into a plurality of subchannels which are connected to the suction orifices 28. The two inputs 25, 26 are each provided with a connector designed to be connected to a vacuum generation means. When the applanation glass is placed on the bottom rim, a vacuum is applied to the two annular channels via the two inputs and generates a suction force which solidly presses the applanation glass against the bottom rim.

Referring to FIG. 5B, when the two parts are assembled together, the top rim 36 of the suction cone 14 is bearing against the base 39 of the truncated cone of the upper part 13. The two parts are fixed together by four screws 35.

According to an embodiment of the disclosure and referring to FIG. 1, the applanation device further comprises two locking rings 16 for locking the applanation body 11 in position on the angular orientation ring 15.

According to an embodiment of the disclosure, the applanation device is produced in a metal material such as Cr-Co by an additive manufacturing technique, called selective or powder bed laser fusion, for issues of technical feasibility on the one hand, and also of price. The blank produced by additive manufacturing is then trimmed by mechanical milling to impose a determined height, and polished by bead-blasting, sand-blasting or manual polishing.

Referring to FIG. 7A and FIG. 7B, when the applanation device is positioned in the suction ring 40 in a configuration ready for the operation, the applanation glass is pressed against the cornea of the eye of the patient to flatten the surface of the eye in order to create a reference plane for the different laser operations to be performed in the cornea. According to an exemplary embodiment illustrated in FIG. 7A, the applanation glass 32, called planar-planar glass, is flat on both of its surfaces. This glass is used for a great majority of operations, such as cutting on a surface or close to the top face of the cornea (examples: LASIK or KLA). It has a thickness of approximately 1 mm for example and a diameter of 14 mm. According to another exemplary embodiment illustrated in FIG. 7B, it can have a flat top surface and a concave bottom surface disposed facing the cornea. The radius of curvature of the concave surface is slightly greater than the average radius of curvature of the cornea. This so-called planar-concave glass 33 is used for more specific operations, such as cutting at depth in order to avoid the formation of folds in the corneal tissue (examples: lenticular ablation, intrastromal pockets, KT or KLP). It has a diameter of 15 mm and a thickness at the center of approximately 2 mm.

According to an embodiment, the applanation glass is provided with a pattern and/or a rule etched on the surface or in the volume to assist in the accurate positioning of the applanation cone with respect to the center of the eye of the patient, and to view the applanation diameter of the eye. Alphanumeric characters can also be added to the marking if necessary. The etchings can be produced by pico or femtosecond laser.

To accurately place the applanation glass with respect to the focal point of the laser, the heightwise dimension of the suction cone will be important to observe. By virtue of the upper part 13 of the applanation body which is translationally movable on the axis Z-Z, it is possible to refine the distance between the optical output of the focusing optical block and the applanation glass, for it to substantially match the working distance of the focusing optical block. In practice, the height of the suction cone must be adjusted so as to position the focal point of the laser beam just under the applanation glass. The tolerance on the positioning of the applanation glass with respect to the focusing optical block is ±10 µm.

As illustrated in FIG. 1, the focusing optical block 105 generally has a substantially cylindrical form, the axis of which is parallel to the axis Z-Z and is centered on the optical axis of symmetry of the cornea 8. The focusing optical block is composed of an assembly of lenses so as to focus the laser beam in the thickness of the cornea to obtain impact points of constant size on the scale of a few microns over a working field with a diameter of at least 10 mm. When the focusing optical block is incorporated in a laser unit, it has a protruding part 106 with respect to the surface of the laser unit. The outer wall of the protruding part 106 of the optical block is provided with an external threading allowing the fixing ring 17, which is provided with a complementary internal threading, to be fixed thereto by screwing.

According to an embodiment and referring to FIG. 8, the applanation device is coupled to the eye via a suction ring 40, the function of which is to guide the applanation cone during the positioning of the applanation glass 12 on the eye. The suction ring is positioned and applied to the eye of the patient previously by the practitioner. The suction ring 40 comprises suction means which make it possible to hold it in position at a corneal part of the eye of the patient. To couple the applanation device to the suction ring, a portion of the lower part of the applanation cone 11 of the applanation device 10 is designed dimensionally and geometrically to be received in the suction ring 40 so as to position the applanation glass 12 in the suction ring in order to be placed in contact against the eye of the patient.

Referring to FIG. 9, the present disclosure also relates to an ophthalmological surgery appliance 100 for performing a cut in an ocular biological tissue, such as a cornea or a lens. It comprises a laser unit 104 designed to deliver a laser beam. This unit comprises a focusing block for focusing the laser beam at a focal point in the ocular biological tissue and an applanation device 10 as described above.

The laser unit 104 also comprises an optical system for displacement of the laser beam. The laser unit is designed to be mounted at the end of an articulated arm 103. The other end of the arm is mounted on a mobile electrotechnical rack 101. Some of the elements of the equipment are housed in the mobile electrotechnical rack 101. The self-balanced articulated arm makes it possible to position the laser unit above the eye of the patient.

The focusing optical block 105 illustrated in more detail in FIG. 1 comprises a protruding lower part provided on its outer wall with a threading and which extends from a bottom surface of the laser unit 104. The applanation device is fixed onto the protruding part of the focusing optical block via the fixing ring 17.

According to an embodiment of the disclosure, the appliance comprises two cameras 107, 108 configured to view the cutting zone, held in position by camera-holding elements 27, 28 fixed onto the outer wall of the applanation body 11. As FIG. 3B illustrates, the two cameras are oriented so as to direct the two imaging beams toward the inside of the suction cone 14 via corresponding apertures 20.

As FIG. 10A illustrates, the two side vision cameras 107, 108, provided with a lighting ring, positioned on either side of the optical axis of symmetry 8 of the focusing block, make it possible to view and illuminate the eye during the operation. The diameter of the image zone, which is approximately 12 mm, is greater than the cutting zone in which the laser spot is displaced. The two cameras make it possible to observe the cutting zone and to follow the cutting process. The two cameras are equipped with a lighting ring and an autofocus. The two cameras are positioned between the focusing optical block and the cornea such that the imaging beam is oriented by an angle of between 45° and 47° with respect to an optical axis of symmetry of the focusing system. These values allow the eye to be viewed over a field of 10.5 mm while taking into account the bulk of the optics and mechanics of the device, of the cameras and of the applanation device.

As FIG. 10B illustrates, the appliance further comprises a centering camera 109 configured for the centering of the optical axis of the focusing optical block with respect to the biological tissue, one end of the centering camera being inserted into the suction cone 14 via one of the two side apertures 20.

The centering camera 109 is a removable camera which is arranged between the focusing optical block and the eye. It generates a visual field which is returned at 90° by a mirror 110 so as to generate a visual field centered on the optical axis 8 of the focusing system, thus making it possible to position the appliance above the eye of the patient. Advantageously, the camera is equipped with a lighting ring generating an annular alignment spot thus allowing the patient to fix a light spot centered on the optical axis of symmetry of the focusing system. It is also equipped with an autofocus. The centering camera 109 is removed from the applanation body to start the operation.

The present disclosure makes it possible to have an applanation device that is efficient and simple to use for coupling the eye of the patient with a laser system to produce cuts of great quality in an ocular biological tissue, such as the cornea or the lens. One of the advantages of the applanation device of the present disclosure consists in using a suction mechanism incorporated in the wall of the applanation cone to securely hold the applanation glass against a surface of the applanation cone. That makes it possible to eliminate the variation of the thickness of the glue used in the known devices which introduces a distance variation between the optical output of the focusing block and the applanation glass in the plane (XY). The absence of the layer of glue between the bearing surface for the positioning of the glass and the applanation glass makes it possible to avoid bad positioning of the applanation glass with respect to the focal point of the laser which could impact the quality of the cut. By virtue of the accuracy and the reproducibility of the positioning of the applanation glass, it is no longer necessary to proceed with a calibration step before the start of the operation in order to know the position of the focal point formed in the ocular tissue. The tolerance on the positioning of the applanation glass with respect to the focusing system is ±10 µm.

A specific combination of several mechanical parts of different functions further offers the possibility of adjusting the distance between the applanation glass and the focusing optical block, in order to make the coupling process the most accurate possible while simplifying the process of coupling between the applanation device and the laser system.

By virtue of the suction mechanism which makes it possible to avoid a permanent fixing of the glass on the cone, the latter is reusable after sterilization; only the glass is consumable.

The applanation device of the present disclosure finally makes it possible to easily incorporate vision cameras and to introduce a vision camera inside the suction cone.

The device of the present disclosure has a relatively low manufacturing cost, because it is produced in a metallic material, for example in Cr—Co, allowing manufacture by additive manufacturing.

### INDUSTRIAL APPLICATION

The applanation device of the disclosure can be used in coupling with different laser systems such as femtosecond or picosecond lasers for the various laser operations to be performed in the cornea and refractive surgery.

What is claimed is:

1. An applanation device configured to be positioned between the eye of a patient and a laser system, to hold the eye in position and create a reference plane for a laser ophthalmological surgery operation, said device comprising:
an applanation cone defining an internal space, a top aperture and a bottom aperture;
said applanation cone comprising an upper part configured to be fixed onto a protruding part of a focusing optical block of the laser system and a lower part comprising suction means;
the bottom aperture being delimited by a bottom rim;
an applanation glass that is transparent to the laser beam generated by the laser system being positioned and held by said suction means against the bottom rim so as to close the bottom aperture.

2. The applanation device as claimed in claim 1, wherein the suction means comprise at least one suction channel extending in the wall of said suction cone between a suction input and a plurality of suction orifices formed in the bottom rim.

3. The applanation device as claimed in claim 1, further comprising a fixing ring configured to be securely mounted on the protruding part of the focusing optical block, the upper part of said applanation cone being configured to be mounted with translational mobility in a direction parallel to an optical axis of the focusing optical block on said fixing ring.

4. The applanation device as claimed in claim 3, further comprising an angular orientation ring configured to be mounted with rotational mobility in a plane at right angles to the optical axis of the focusing optical block on said protruding part, the upper part of said applanation cone being configured to be mounted with translational mobility in a direction parallel to the optical axis of the focusing optical block on said angular orientation ring.

5. The applanation device as claimed in claim 1, wherein the applanation cone comprises at least one camera-holding element fixed onto the outer wall of the applanation cone and at least one side aperture arranged with respect to said holding element so as to allow the passage of an imaging beam generated by said at least one camera to view a cutting zone of the eye situated under the applanation glass.

6. The applanation device as claimed in claim 5, wherein said holding element is configured such that the imaging beam generated by said camera is inclined by an angle of between 30° and 50°, preferably between 45° and 47°, with respect to the optical axis of the focusing optical block.

7. The applanation device as claimed in claim 1, wherein the applanation glass comprises at least one pattern or a graduated rule produced on the surface or in the volume.

8. The applanation device as claimed in claim 1, wherein the applanation glass has a planar-planar or planar-concave geometry.

9. The applanation device as claimed in claim 3, wherein the upper part of said applanation cone comprises an axial displacement ring provided with an inner threading, designed to be engaged by screwing with an outer threading produced on the outer wall of the fixing ring or of the orientation ring, said axial displacement ring ensuring, in a screwing step, an axial displacement of said applanation cone in a direction parallel to the optical axis of the focusing optical block.

10. The applanation device as claimed in claim 3, further comprising a locking ring configured to lock the applanation cone in position.

11. The applanation device as claimed in claim 1, wherein the applanation cone is produced in a metallic material by additive manufacturing.

12. The applanation device as claimed in claim 1, wherein the applanation cone comprises a lower part fixed onto the upper part, said lower part having an upside-down truncated cone form.

13. An applanation assembly for holding an eye of a patient in position and creating a reference plane for a laser system for a laser ophthalmological surgery operation, said assembly comprising:
an applanation device as claimed in claim 1;
a suction ring designed to come into contact with a surface of the eye of the patient;
a portion of said lower part of the applanation cone being configured to be inserted into the suction ring to position the applanation glass inside the suction ring.

14. An ophthalmological surgery appliance for making a cut in an ocular biological tissue, such as a cornea or a lens, comprising:
a laser unit designed to deliver a laser beam,
a focusing optical block for focusing the laser beam at a focal point in the ocular biological tissue;
said focusing optical block comprising a protruding lower part extending from a bottom surface of the laser unit,
an applanation device as claimed in claim 1 configured to be fixed onto the protruding lower part of the focusing optical block so as to position the applanation glass at a distance substantially close to the focusing distance with respect to the optical output of the focusing optical block.

15. The appliance as claimed in claim 14, further comprising at least one camera configured to view the cutting zone, said at least one camera being held in position on the outer wall of the applanation cone.

16. The appliance as claimed in claim 14, further comprising a centering camera configured for the centering of the optical axis of symmetry of the focusing system with respect to the biological tissue, one end of the centering camera being inserted into the applanation cone via one of the two apertures provided in the applanation cone.
